(19) United States
(12) Patent Application Publication
(10) Pub. No.: US 2025/0270185 A1
(43) Pub. Date: Aug. 28, 2025

(54) METHOD FOR PREPARATION OF IPTACOPAN HYDROCHLORIDE

(71) Applicant: HANGZHOU CHEMINSPIRE TECHNOLOGIES CO., LTD., Hangzhou (CN)

(72) Inventors: Yiping ZHANG, Hangzhou (CN); Qiaoling LIU, Hangzhou (CN); Yihua WU, Hangzhou (CN); Xuchun ZHENG, Hangzhou (CN)

(73) Assignee: HANGZHOU CHEMINSPIRE TECHNOLOGIES CO., LTD., Hangzhou (CN)

(21) Appl. No.: 19/067,741

(22) Filed: Feb. 28, 2025

(30) Foreign Application Priority Data
Feb. 28, 2024 (CN) 202410220675.0

(51) Int. Cl. C07D 401/06; C07D 209/42; C07D 211/48; C12N 9/04

(52) U.S. Cl. CPC C07D 401/06; C07D 209/42; C07D 211/48; C12N 9/0006; C12Y 101/01

(57) ABSTRACT

A synthesis method for iptacopan is provided, which includes the asymmetric addition of compound 1 as the starting material with compound 2 benzyl 4-oxo-3,4-dihydropyridine-1(2H)-carboxylate under the action of a metal catalyst and ligand to obtain intermediate compound 3; then, carbonyl group being enzymatically reduced to obtain compound 4; compound 4 undergoing catalytic conversion from the cyano group to the carboxyl group under the action of a cyano hydrolysis enzyme in a one-pot reaction; subsequently, hydroxy groups completing the ethylation reaction; after palladium hydrogenation, key intermediate compound 6 of iptacopan being obtained; finally, compounds 6 and 7 undergoing reduction amination condensation, followed by removing the Boc group to form the target product: hydrochloride salt of iptacopan. This method features a simple process route, high yield, and low costs, which is suitable for industrial production.

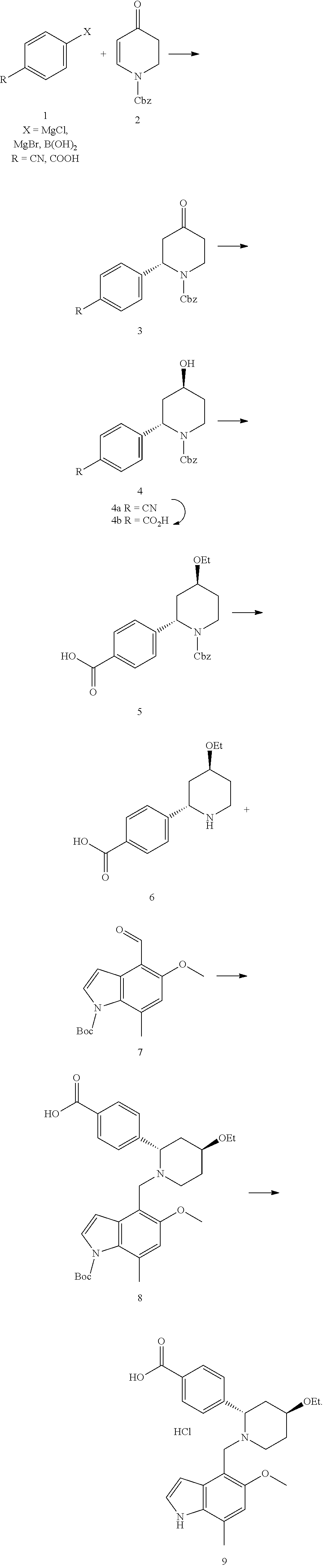

### CROSS-REFERENCE TO RELATED APPLICATIONS

This application claims priority to Chinese Patent Application No. 202410220675.0, filed on Feb. 28, 2024, the contents of which are hereby incorporated by reference to its entirety.

### TECHNICAL FIELD

The present disclosure relates to the field of pharmaceutical Chemicals, and specifically involves a synthesis method of iptacopan hydrochloride and its intermediates as a reversible complement regulator B (CFB) target inhibitor for the treatment of adult paroxysmal nocturnal hemoglobinuria.

### BACKGROUND

Iptacopan is an orally administered, highly selective, small-molecule reversible complement regulator B (CFB) inhibitor Invented by Novartis Pharmaceuticals. FB is a key serine protease in the alternative pathway of the complement system. Iptacopan acts upstream in the C5-terminal pathway of the complement system, blocking hemolytic PNH in both intravascular hemolysis (IVH) and extravascular hemolysis (EVH). It may treat diseases caused by dysfunction in multiple alternative pathways without affecting the immune response mediated by other complement pathways against microbial invasion, thereby reducing the risk of infection for patients. In December 2023, the U.S. Food and Drug Administration (FDA) approved the oral drug iptacopan (brand name Fabhalta) for the treatment of paroxysmal nocturnal hemoglobinuria in adults. Its approval and market launch in China are also in progress, with a promising market outlook for its active pharmaceutical ingredients and related intermediates.

The chemical name of iptacopan API is 4-(2S,4S)-4-ethoxy-1-(5-methoxy-7-methyl-1H-indole-4-yl)methyl) piperidine-2-yl)benzoate hydrochloride, and its structural formula is as follows:

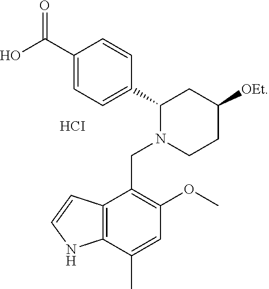

Novartis patent WO2015009616 reports the compound iptacopan and its synthesis method, using 4-bromobenzonitrile and 4-methoxypyridine as starting materials. After the Grignard reagent exchange of 4-bromobenzonitrile, it first undergoes an addition reaction with 4-methoxypyridine under the action of benzyl chloroformate to form 2-(4-cyano-phenyl)-4-oxo-3,4-dihydropyridine-1 (2H)-carboxylate. Then, the double bond is reduced with zinc powder, and the carbonyl group is reduced with lithium borohydride, then the hydroxyl group is protected with TBDPSCl. After column separation, the relative configuration (2S,4S)-benzyl 4-(tert-butyldiphenylsilyl oxy)-2-(4-cyano-phenyl) piperidine-1-carboxylate is obtained. This intermediate is then deprotected with TBAF to expose the hydroxyl group, followed by iodoethane protection of the hydroxyl group to complete the etherification. The optically pure (2S,4S)-configuration intermediate is obtained through separation using a chiral column. Subsequently, the cyano group is hydrolyzed to obtain a carboxylic acid intermediate, which is then methylated and hydrogenated using palladium on carbon to obtain the key intermediate 4-((2S,4S)-4-Ethoxypiperidin-2-yl)benzoic acid methyl ester. This intermediate and another key intermediate of iptacopan, tert-butyl 4-formyl-5-methoxy-7-methyl-1H-indole-1-carboxylate, undergo a reductive amination reaction under the action of sodium triacetoxyborohydride. Finally, the ester is hydrolyzed and the Boc protecting group is removed to obtain the target compound iptacopan.

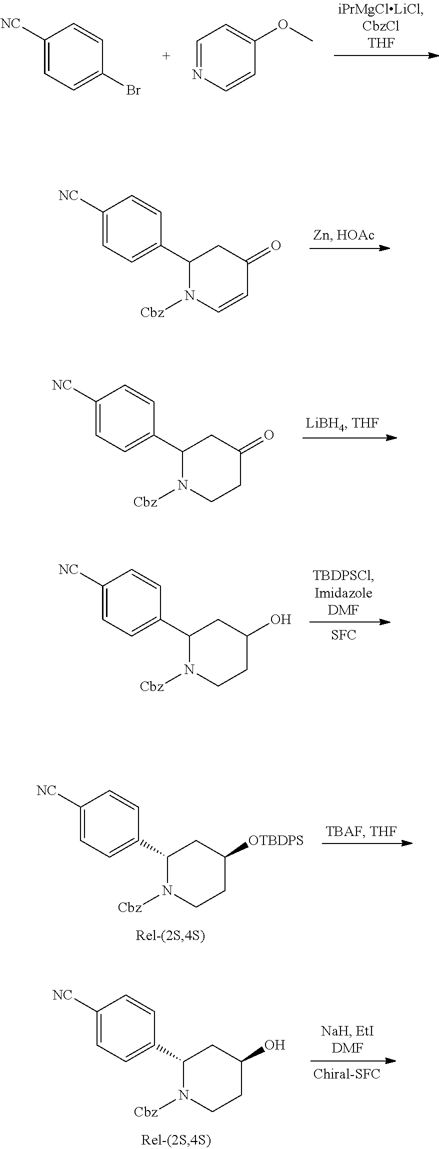

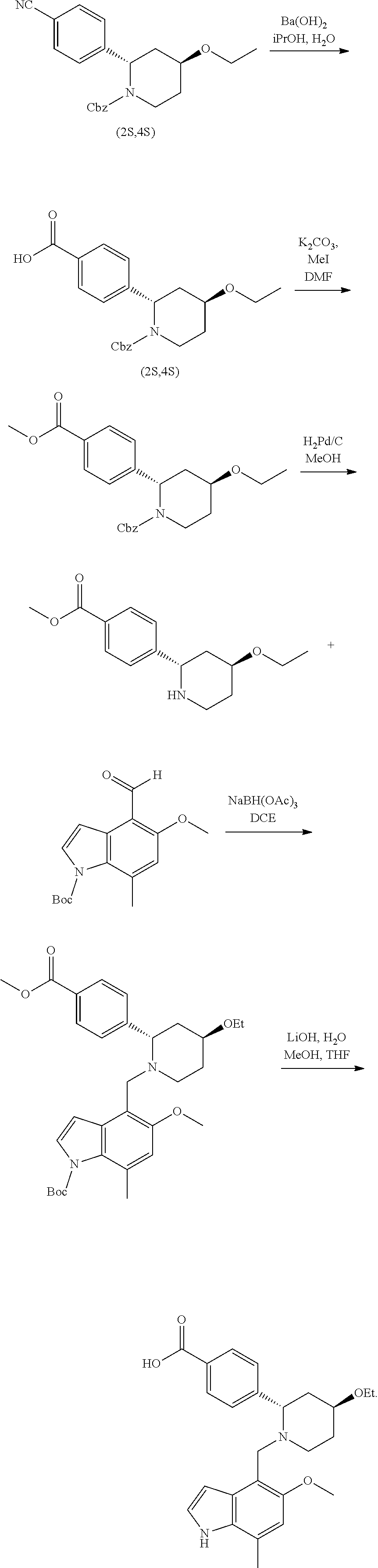

Novartis patent WO2020016749 improved the synthesis method of iptacopan, using 4-methoxypyridine to obtain 4-oxo-3,4-dihydropyridine-1 (2H)-benzyl formate under the action of benzyl chloroformate and sodium borohydride. Then, under the catalysis of rhodium metal and phosphine ligand, it undergoes asymmetric addition of a double bond with 4-methoxycarbonylphenylboronic acid to form(S)-benzyl 2-(4-(methoxycarbonyl)phenyl)-4-oxopiperidine-1-carboxylate. The carbonyl group is then reduced using enzymatic catalysis, followed by protection of the hydroxyl group with TBS. Then, it undergoes a hydroxyethylation reaction with metaldehyde in the system of triethylsilane and triethylsilyl trifluoromethanesulfonate. Palladium hydrogenation removes the Cbz protection and forms a salt with maleic acid to obtain the key intermediate 4-((2S, 4S)-4-Ethoxypiperidin-2-yl)benzoic acid methyl ester maleate. Finally, this intermediate reacts with another key intermediate of iptacopan, 4-formyl-5-methoxy-7-methyl-1H-indole-1-carboxylic acid tert-butyl ester, under the action of an iridium catalyst using high-pressure hydrogen and carbon monoxide reduction to obtain the Boc methyl ester intermediate N1 of iptacopan. This method still has certain drawbacks; the asymmetric addition reaction requires cryogenic conditions −70° C. and has a low yield, the catalyst rhodium is expensive and the equivalent used is large, the subsequent hydroxy ethylification reaction is complicated, and the total yield is low, so the route cost is still high.

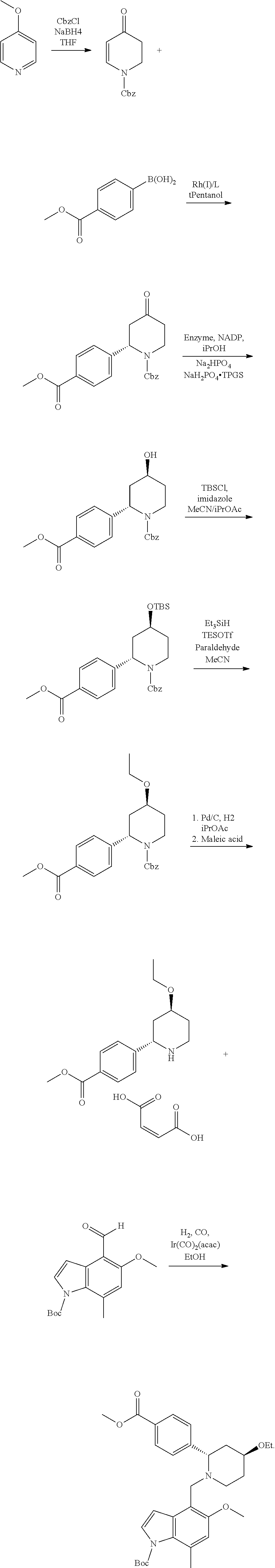

In short, the existing methods for synthesizing iptacopan involve multiple steps, which pose challenges for large-scale production, difficult process scale-up, low yield, and high process cost. Therefore, it is still necessary to find a synthesis method with a simple process route, low cost, and suitable for industrial production.

### SUMMARY

In view of the deficiencies of existing technologies, the purpose of this disclosure is to provide a preparation method of iptacopan. The preparation process route of this disclosure is simple, with high yield, and low cost and it is suitable for industrial production.

One of the purposes of the present disclosure is to provide a synthesis method of iptacopan, and the present disclosure adopts the following technical solution.

A process for the preparation of iptacopan comprises the following steps:
(a) the compound 6 and the compound 7 undergoing a reductive amination reaction to get the intermediate compound 8;

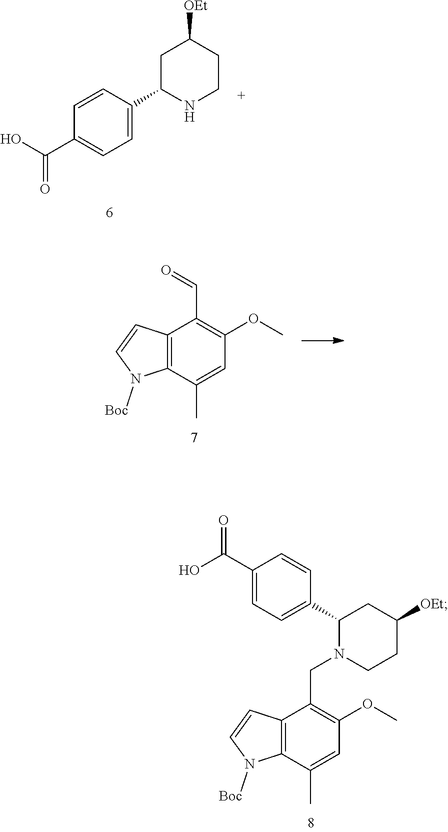 [SMILES: CCO[C@H]1CCN[C@H](c2ccc(C(=O)O)cc2)C1 ; COc1cc(C)c2c(ccn2C(=O)OC(C)(C)C)c1C=O ; CCO[C@H]1CCN(Cc2c(OC)cc(C)c3c2ccn3C(=O)OC(C)(C)C)[C@H](c2ccc(C(=O)O)cc2)C1]

(b) the compound 8 undergoing a de-protection reaction to remove the Boc protection group and a salification reaction with hydrochloric acid to obtain the compound 9 Iptacopan hydrochloride;

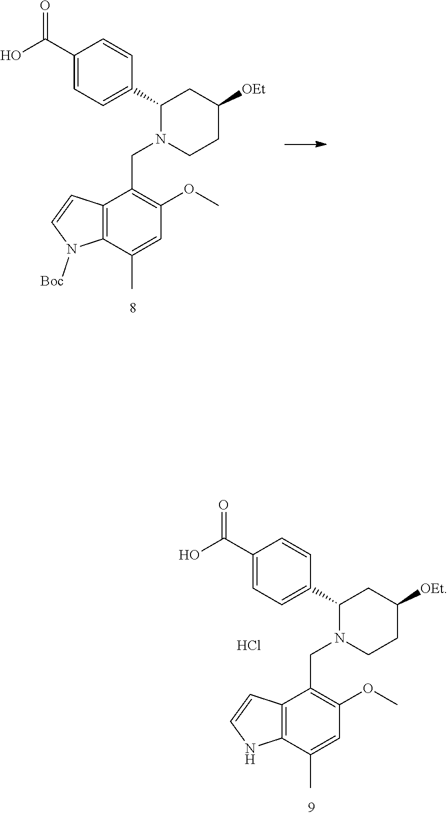 [SMILES: CCO[C@H]1CCN(Cc2c(OC)cc(C)c3c2ccn3C(=O)OC(C)(C)C)[C@H](c2ccc(C(=O)O)cc2)C1 ; CCO[C@H]1CCN(Cc2c(OC)cc(C)c3[nH]ccc23)[C@H](c2ccc(C(=O)O)cc2)C1 ; Cl]

As a preferred option, in the reductive amination reaction of step (a), a reducing reagent is selected from sodium borohydride, lithium borohydride, potassium borohydride, sodium triacetoxyborohydride, sodium cyanoborohydride, triethylsilane, or 1,1,3,3-tetramethyldisiloxane; without adding Lewis acid or with the addition of a Lewis acid, wherein the Lewis acid is selected from acetic acid, titanium tetrachloride, or tetraisopropyl titanate; the reaction solvent is selected from dichloromethane, 1,2-dichloroethane, acetonitrile, tetrahydrofuran, 1,4-dioxane, or toluene; the reaction temperature ranges from −10° C. to 90° C.

As a preferred option, in the reaction of step (b), the target product compound 9 is obtained in three ways including adding hydrochloric acid to perform the de-protection reaction by removing Boc protection group and the salification reaction in one-pot reaction, or by directly removing Boc protection without the acid and then performing the salification reaction with hydrochloric acid, or by removing Boc protection with an organic acid and then performing salification reaction with hydrochloric acid; the organic acid is selected from acetic acid, trifluoroacetic acid, citric acid, tartaric acid, methanesulfonic acid, p-toluenesulfonic acid, or trifluoromethanesulfonic acid; the reaction solvent is selected from methanol, ethanol, isopropanol, n-butanol, tert-butanol, isopentanol, tert-pentanol, ethyl acetate, isopropyl acetate, tetrahydrofuran, 2-methyltetrahydrofuran, 1,4-dioxane, methyl tert-butyl ether, isopropyl ether, acetone, acetonitrile, toluene, or water, or any mixture of two of these solvents. The reaction temperature ranges from −20° C. to 120° C.

The second purpose of the present disclosure is to provide a synthesis method for intermediate compound 6 of iptacopan, comprising the following technical solution.

A process for the preparation of the intermediate compound 6 of iptacopan comprises the following steps.
(a) the compound 4 undergoing an alkylation reaction with an ethylating reagent in the presence of base to obtain the compound 5;

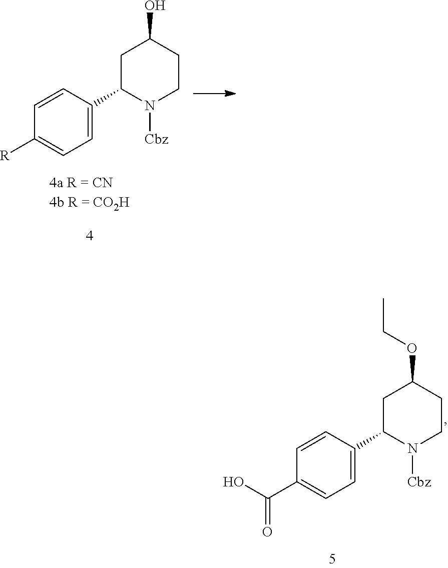

(b) the compound 5 undergoing a hydrogenation reaction under palladium catalyst to remove Cbz protection group to obtain the intermediate compound 6;

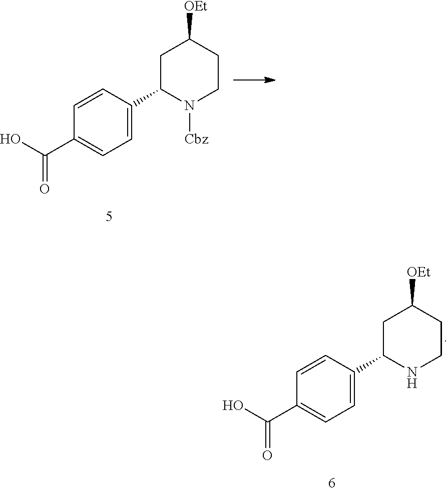 [SMILES: CCO[C@H]1CCN(C(=O)OCc2ccccc2)[C@H](c2ccc(C(=O)O)cc2)C1 ; CCO[C@H]1CCN[C@H](c2ccc(C(=O)O)cc2)C1]

As the preferred option, in the alkylation reaction of step (a), the ethylating reagent is selected from diethyl sulfate, diethyl carbonate, ethyl iodide, ethyl bromide, ethyl methanesulfonate, or ethyl p-toluenesulfonate; the base is selected from sodium methoxide, sodium ethoxide, potassium tert-butoxide, sodium tert-butoxide, LiHMDS, KHMDS, sodium hydroxide, lithium hydroxide, or potassium hydroxide; without adding a catalyst or with the addition of a catalyst, wherein the catalyst is selected from potassium iodide, sodium iodide, tetrabutylammonium bromide, tetrabutylammonium hydroxide, trimethylbenzyl ammonium chloride, or triethylbenzyl ammonium chloride; the reaction solvent is selected from DMSO, toluene, NMP, dichloromethane, tetrahydrofuran, 2-methyltetrahydrofuran, or any mixture of two of these solvents or water; the reaction temperature ranges from −10° C. to 11° C.

Preferably, in the compound 4, R is CN, the cyanide hydrolysis reaction is directly performed by adding water and heating or heating in the original alkaline aqueous mixture, and in the compound 5, R is COOH.

As a preferred option, in step (b), the palladium catalyst is selected from palladium on carbon or palladium hydroxide; the reaction solvent is selected from methanol, ethanol, isopropanol, tetrahydrofuran, ethyl acetate, or isopropyl acetate.

The third purpose of the present disclosure is to provide a synthesis method of the intermediate compound 4 of iptacopan, which adopts the following technical solution.

A process for the preparation of the intermediate compound 4 of iptacopan comprises the following steps.
(a) the compound 1 and the compound 2 undergoing an asymmetric addition to obtain the intermediate compound 3 under the catalysis of a chiral phosphine ligand metal complex;

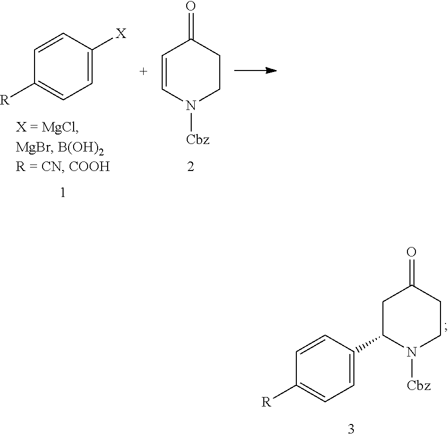

(b) the compound 3 undergoing a carbonyl reduction reaction to obtain the intermediate compound 4 under the catalysis of a reductase;

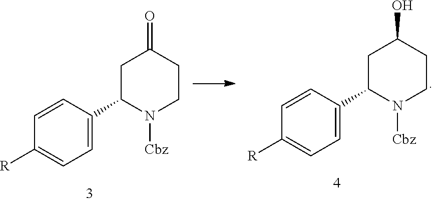

As a preferred option, when R is CN, it is preferable that carbonyl group is first reduced under the catalysis of a reductase, followed by the one-pot addition of a cyano hydrolysis enzyme to hydrolyze the cyano group into a carboxylic acid:

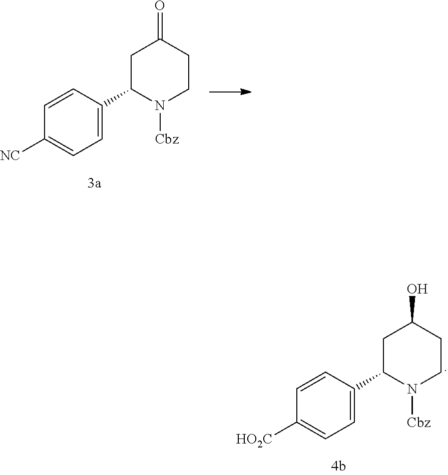 [SMILES: N#Cc1ccc([C@@H]2CC(=O)CCN2C(=O)OCc2ccccc2)cc1 ; O=C(O)c1ccc([C@@H]2C[C@@H](O)CCN2C(=O)OCc2ccccc2)cc1]

As a preferred option, in the reaction of step (a), the chiral phosphine ligand metal complex catalyst is a complex formed by a metal catalyst and a chiral phosphine ligand, which is used directly or generated in situ to participate in the reaction. The metal catalyst is selected from cuprous bromide, cuprous iodide, cuprous chloride, palladium acetate, palladium trifluoroacetate, palladium dichloride, sodium tetrachloropalladate, chlorobis (ethylene) rhodium (I) dimer, or bis(1,5-cyclooctadiene) rhodium(I) tetrafluoroborate.

As a preferred option, When X is MgCl or MgBr, the compound 1 is obtained by the exchange of 4-bromobenzoic acid, 4-iodobenzoic acid, 4-bromobenzonitrile, or 4-iodobenzonitrile with isopropyl magnesium chloride, isopropylmagnesium chloride-lithium chloride complex, or isopropyl magnesium bromide, or any combination of these with n-butyl lithium; the catalyst is a complex formed in situ from copper metal catalyst and chiral phosphine ligand; the copper metal catalyst is selected from cuprous bromide, cuprous iodide, or cuprous chloride, and the preferred chiral phosphine ligand is selected from 1,2-bis((2S,5S)-2,5-diethylphospholane-1-yl)benzene, S-(−)-2,2′-bis(diphenylphosphino)-1,1′-binaphthalene, 1,2-bis((2S,5S)-2,5-dimethylphospholane)benzene, 1,2-bis[(2S,5S)-2,5-diethylphospholane]benzene, 1,2-bis[(2S,5S)-2,5-dimethylphospholane]ethane, 1,2-bis((2S,5S)-2,5-diphenylphospholane) ethane or (S,S)-bis[(2-methoxyphenyl)phenylphosphino]ethane; the reaction solvent is selected from dichloromethane, 1,2-dichloroethane, toluene, tetrahydrofuran, or 2-methyltetrahydrofuran, or any mixture of two of these solvents; the reaction temperature ranges from −80° C. to 60° C.

As a preferred option, When X is B(OH)₂, the preferred catalyst is selected from (1,2-bis((2S,5S)-2,5-diethylphosphorazepane-1-yl)benzene) (1,5-cyclooctadiene) trifluoromethanesulfonate rhodium(I), S-(+)-2,2-bis(diphenylphosphine)-1,1-bis-naphthalene (1,5-cyclooctadiene) tetrafluoroborate rhodium, 1,2-bis[(2S,5S)-2,5-diethylphosphonyl]benzene (1,5-cyclooctadiene) tetrafluoroborate rhodium(I), 1,2-bis[(2S,5S)-2,5-dimethylphosphorazepane-1,3-phenylene]benzene (cyclooctadiene) tetrafluoroborate rhodium(I), or (S,S)-(+)-1,2-bis[(2-methoxyphenyl)(phenyl)phosphine]ethane (1,5-cyclooctadiene) tetrafluoroborate rhodium(I); the reaction solvent is selected from methanol, ethanol, isopropanol, n-butanol, tert-butanol, n-pentanol, tert-pentanol, acetonitrile, tetrahydrofuran, 2-methyltetrahydrofuran, ethyl acetate or isopropyl acetate; and the reaction temperature ranges from −80° C. to 60° C.

As a preferred option, in the reaction of step (b), the reductase is selected from ketoreductase KRED, alcohol dehydrogenase, isopropanol dehydrogenase, or glucose dehydrogenase (GDH), or any combination of two of these; the ketoreductase is selected from KRED-EW124, KRED-101, KRED-MY236, or KRED-MY352; and the coenzyme used is selected from nicotinamide adenine dinucleotide (NAD), nicotinamide adenine dinucleotide phosphate (NADP), flavin adenine dinucleotide (FAD), pyridoxal monophosphate (PLP), or mixtures of them; the reducing reagent is selected from isopropanol, ethanol, or glucose; the reaction solvent is selected from dimethyl sulfoxide (DMSO), N-methylpyrrolidone, acetonitrile, ethyl acetate, isopropyl acetate, water or any mixture of two of these solvents; the buffer reagent is selected from phosphoric acid, potassium dihydrogen phosphate, dipotassium hydrogen phosphate, disodium hydrogen phosphate, monosodium dihydrogen phosphate, tris (hydroxymethyl) aminomethane hydrochloride, or any mixture buffer solution of two of these reagents; and the reaction temperature ranges from 0° C. to 60° C.

Preferably, when R is CN, the described one-pot cyano hydrolysis reaction involves adjusting the reaction mixture to a pH of 5-9 using PBS buffer solution after the carbonyl reduction reaction is complete. Cyano hydrolysis enzyme is then added to convert the cyano group into a carboxylic acid group in one pot. The cyano hydrolysis enzyme used is selected from NIT83, NIT101, NIT139, SP409, NIT-MY20, or NIT-MY25; and the reaction temperature ranges from 20° C. to 50° C.

Specifically, a process for preparation of iptacopan includes the asymmetric addition of compound 1 as the starting material with the compound 2 benzyl 4-oxo-3,4-dihydropyridine-1 (2H)-carboxylate under the catalysis of a metal catalyst and ligand to obtain the intermediate compound 3, followed by enzymatic reduction of the carbonyl group to obtain the compound 4; the compound 4 undergoing a one-pot cyano hydrolysis catalyzed by an enzyme to complete the conversion of the cyano group to the carboxylic acid group; subsequently, hydroxy group undergoing an ethylation reaction, followed by palladium hydrogenation to obtain the key intermediate compound 6 of iptacopan; finally, the compound 6 and 7 undergoing a reductive amination, followed by removing Boc group to form the iptacopan hydrochloride salt. The reaction route is as follows:

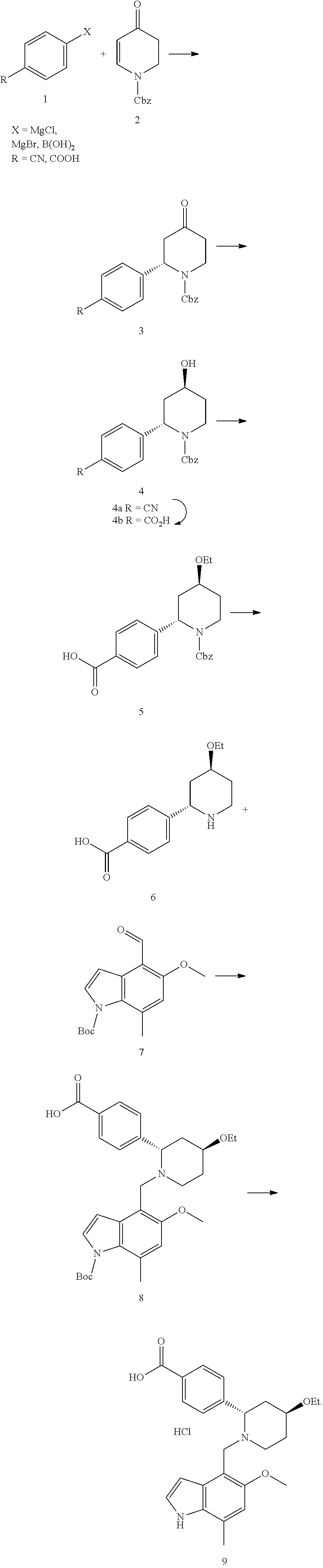

The synthesis method of iptacopan hydrochloride provided by this method is simple, high yield, and low cost, which is suitable for industrial production.

### DETAILED DESCRIPTION

The embodiments of the present disclosure are described in detail below. The embodiments are implemented on the premise of the technical solution of the present disclosure, and detailed implementation methods and specific operation processes are provided, but the scope of protection of the present disclosure is not limited to the embodiments described below.

### Example 1

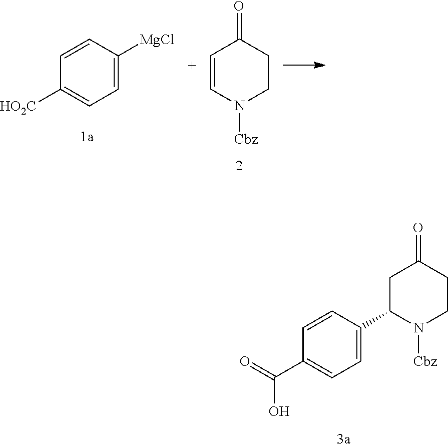

In a reaction flask A, 4-bromobenzoic acid (24.12 g, 120 mmol) and tetrahydrofuran (230 mL) are added. The mixture is cooled to 0-5° C. under nitrogen protection, add dropwise 1.3M solution of isopropylmagnesium chloride-lithium chloride in tetrahydrofuran (264 mmol, 203 mL), stir at this temperature for 2 hours to obtain a tetrahydrofuran solution of 1a. In another reaction flask B, add compound 2 (23.12 g, 100 mmol), tetrahydrofuran (115 mL), and 1,2-bis((2S,5S)-2,5-diphenylphospholane) ethane (0.5 mmol, 253 mg). After switching to nitrogen under vacuum three times, cool to 0-5° C., add cuprous bromide (0.5 mmol, 72 mg) under nitrogen protection and stir for 1 hour. Slowly drip the prepared tetrahydrofuran solution of 1a from reaction flask A into the mixture of reaction flask B with a peristaltic pump, and keep the temperature at 0-5° C. after dripping, stir for 4-6 hours. After the reaction is complete, 5% dilute hydrochloric acid solution (230 mL) is added to quench the reaction, ethyl acetate (115 mL) is added and extracted twice, the organic phase is washed once with water (115 mL), concentrated to a small volume, n-heptane (230 mL) is slowly added, and cool slowly to 0-5° C. After crystallization, filter the mixture to obtain the solid intermediate compound 3a (31.33 g, a yield of 88.4%, a purity of 99.7%, ee=99.8%).

In Example 1, 4-bromobenzoic acid may be replaced by 4-iodobenzoic acid, 4-bromobenzonitrile, or 4-iodobenzonitrile; isopropylmagnesium chloride-lithium chloride may be replaced by isopropyl magnesium chloride or isopropyl magnesium bromide; cuprous bromide may be replaced by cuprous iodide or cuprous chloride; the chiral phosphine ligand 1,2-bis((2S,5S)-2,5-diphenylphospholane) ethane may be replaced by 1,2-bis((2S,5S)-2,5-diethylphospholane-1-yl)benzene, S-(−)-2,2′-bis(diphenylphosphino)-1,1′-binaphthalene, 1,2-bis((2S,5S)-2,5-dimethylphospholane)benzene, 1,2-bis[(2S,5S)-2,5-diethylphospholane]benzene, 1,2-bis[(2S,5S)-2,5-(dimethylphospholane)]ethane, or (S,S)-bis[(2-methoxyphenyl)phenylphosphino]ethane; the solvent tetrahydrofuran may be replaced by dichloromethane, 1,2-dichloroethane, toluene, or 2-methyltetrahydrofuran, or any mixture of two of these.

### Example 2

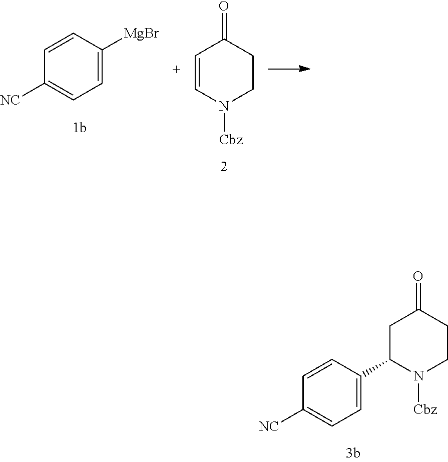

In a reaction flask A, 4-bromobenzonitrile (21.84 g, 120 mmol) and tetrahydrofuran (230 mL) are added, and the mixture is cooled to 0-5° C. under nitrogen protection, add 2M solution of isopropyl magnesium bromide in tetrahydrofuran (132 mmol, 66 mL), and stir at room temperature for 0.5 hour to obtain a tetrahydrofuran solution 1b. In another reaction flask B, add compound 2 (23.12 g, 100 mmol), dichloromethane (115 mL), and (S,S)-bis[(2-methoxyphenyl)phenylphosphino]ethane (0.5 mmol, 229 mg). After switching to nitrogen under vacuum three times, cool to 0-5° C., add cuprous bromide (0.5 mmol, 72 mg) under nitrogen protection and stir for 1 hour. Slowly drip the prepared tetrahydrofuran solution of 1b from reaction flask A into the mixture of reaction flask B with a peristaltic pump, and keep the temperature at 0-5° C. after dripping, stir for 4-6 hours. After the reaction is complete, add 5% dilute hydrochloric acid solution (230 mL) to quench the reaction. Add dichloromethane (115 mL) for two times extraction, combine the organic phase and wash with water (115 mL) once, concentrate to a small volume, slowly add petroleum ether (230 mL), and slowly cool to 0-5° C. After crystallization, filter the mixture to obtain the pale solid intermediate compound of formula 3b (30.60 g, a yield of 91.2%, a purity of 99.6%, ee=99.7%).

In Example 2, 4-bromobenzonitrile may be replaced by 4-bromobenzoic acid, 4-iodobenzoic acid, or 4-iodobenzonitrile; isopropyl magnesium bromide may be replaced by isopropylmagnesium chloride-lithium chloride or isopropyl magnesium chloride; cuprous bromide may be replaced by cuprous iodide or cuprous chloride; the chiral phosphine ligand (S,S)-bis[(2-methoxyphenyl)phenylphosphino]ethane may be replaced by 1,2-bis((2S,5S)-2,5-diphenylphospholane) ethane, 1,2-bis((2S,5S)-2,5-diethylphospholane-1-yl)benzene, S-(−)-2,2′-bis(diphenylphosphino)-1,1′-binaphthalene, 1,2-bis((2S,5S)-2,5-dimethylphospholane)benzene, 1,2-bis[(2S,5S)-2,5-diethylphospholane]benzene, or 1,2-bis[(2S,5S)-2,5-(dimethylphospholane)]ethane, the solvent tetrahydrofuran may be replaced by dichloromethane, 1,2-dichloroethane, toluene, or 2-methyltetrahydrofuran, or any mixture of two of these; dichloromethane may be replaced by tetrahydrofuran, 1,2-dichloroethane, toluene, or 2-methyltetrahydrofuran, or any mixture of two of these.

### Example 3

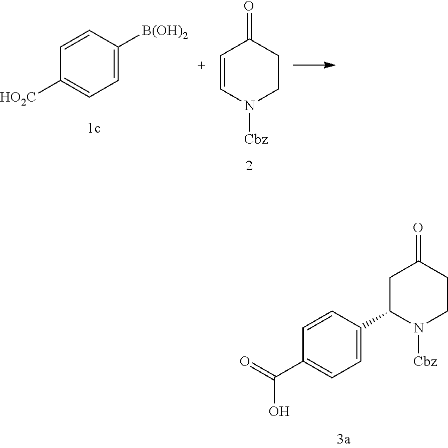 [SMILES: O=C(O)c1ccc(B(O)O)cc1 ; O=C1C=CN(C(=O)OCc2ccccc2)CC1 ; O=C1CCN(C(=O)OCc2ccccc2)[C@H](c2ccc(C(=O)O)cc2)C1]

Compound 2 (23.12 g, 100 mmol) and isopentyl alcohol (115 mL) are added to the reaction flask. After switching to nitrogen under vacuum three times, the mixture is cooled to (−15° C.)-(−5° C.) under nitrogen protection. Add the catalyst 1,2-bis[(2S,5S)-2,5-diethylphosphonyl]benzene (1,5-cyclooctadiene) tetrafluoroborate rhodium(I) (0.2 mmol, 132 mg). After stirring evenly, slowly drip the 4-carboxyphenylboronic acid 1c (19.91 g, 120 mmol dissolved in 115 mL isopentyl alcohol) solution into the reaction flask using a peristaltic pump. After the addition is complete, keep the temperature at (−15° C.)-(−5° C.) and stir for 3 to 4 hours. After the reaction is complete, add 5% dilute hydrochloric acid solution (230 mL) to quench the reaction, separate the phases, and extract the aqueous phase twice with ethyl acetate (115 mL). Combine the organic phases and wash once with water (115 mL), concentrate to a small volume, slowly add n-heptane (230 mL), and cool slowly to 0-5° C. After crystallization, filter the mixture to obtain the solid intermediate compound 3a (32.02 g, yield 90.5%, purity 99.8%, ee=99.8%).

In Example 3, the 1,2-bis[(2S,5S)-2,5-diethylphosphonyl]benzene (1,5-cyclooctadiene) tetrafluoroborate rhodium(I) may be replaced by (1,2-bis((2S,5S)-2,5-diethylphosphorazepane-1-yl)benzene) (1,5-cyclooctadiene) trifluorosulfonate rhodium(I), S-(+)-2,2-bis(phenylphosphine)-1,1-biphenyl (1,5-cyclooctadiene) tetrafluoroborate rhodium, 1,2-Bis [(2S,5S)-2,5-dimethylphospholanyl] benzene (cyclooctadiene) rhodium (I) tetrafluoroborate, or (S,S)-(+)-1,2-bis[(2-methoxyphenyl)(phenyl)phosphine]ethane (1,5-cyclooctadiene) tetrafluoroborate rhodium(I) or Rh(COD)(S,S-DIPAMP)BF₄; The reaction solvent of isopentyl alcohol may be replaced by methanol, ethanol, isopropanol, n-butanol, tert-butanol, n-pentanol, tert-pentanol, acetonitrile, tetrahydrofuran, 2-methyltetrahydrofuran, ethyl acetate or isopropyl acetate.

### Example 4

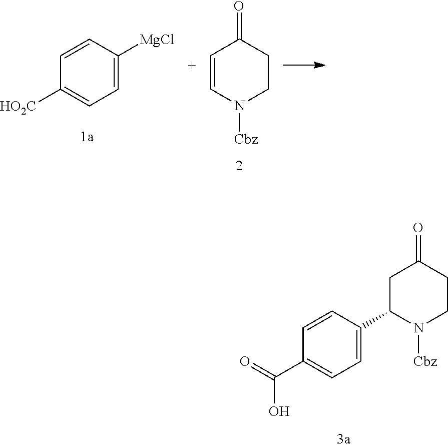

In a reaction flask A, 4-bromobenzoic acid (24.12 g, 120 mmol) and tetrahydrofuran (230 mL) are added. The mixture is cooled to 0-5° C. under nitrogen protection, add dropwise 1.3M solution of isopropylmagnesium chloride-lithium chloride in tetrahydrofuran (180 mmol, 138.5 mL), then slowly add 2.5M n-butyl lithium in hexane solution (48.0 mL, 120 mmol), and stir at this temperature for 2 hours to obtain tetrahydrofuran solution of compound 1a. In another reaction flask B, add compound 2 (23.12 g, 100 mmol), tetrahydrofuran (115 mL), and (S,S)-bis[(2-methoxyphenyl)phenylphosphino]ethane (0.5 mmol, 229 mg). After switching to nitrogen under vacuum three times, cool to 0-5° C., add cuprous bromide (0.5 mmol, 72 mg) under nitrogen protection and stir for 1 hour. Slowly drip the prepared tetrahydrofuran solution of 1a in reaction flask A into the mixture in reaction flask B with peristaltic pump. After dripping, keep it at 0-5° C. and stir for 4-6 hours. After the reaction is complete, add 5% dilute hydrochloric acid solution (230 mL) to quench the reaction. Add ethyl acetate (115 mL) and extract twice. The organic phase is washed once with water (115 mL) and concentrated to a small volume. Slowly add n-heptane (230 mL), and cool slowly to 0-5° C. After crystallization, filter the mixture to obtain the solid intermediate compound formula 3a (31.94 g, a yield of 90.2%, a purity of 99.8%, ee=99.8%).

In Example 4, 4-bromobenzoic acid may be replaced by 4-iodobenzoic acid, 4-bromobenzonitrile, or 4-iodobenzonitrile; isopropylmagnesium chloride-lithium chloride may be replaced by isopropyl magnesium chloride or isopropyl magnesium bromide; cuprous bromide may be replaced by cuprous iodide or cuprous chloride; the chiral phosphine ligand (S,S)-bis[(2-methoxyphenyl)phenylphosphino]ethane may be replaced by 1,2-bis((2S,5S)-2,5-diphenylphospholane) ethane, 1,2-bis((2S,5S)-2,5-diethylphospholane-1-yl)benzene, S-(−)-2,2′-bis(diphenylphosphino)-1,1′-binaphthalene, 1,2-bis((2S,5S)-2,5-dimethylphospholane)benzene, 1,2-bis[(2S,5S)-2,5-diethylphospholane]benzene, or 1,2-bis[(2S,5S)-2,5-(dimethylphospholane)]ethane; the solvent tetrahydrofuran may be replaced by dichloromethane, 1,2-dichloroethane, toluene, or 2-methyltetrahydrofuran, or any mixture of two of these solvents.

### Example 5

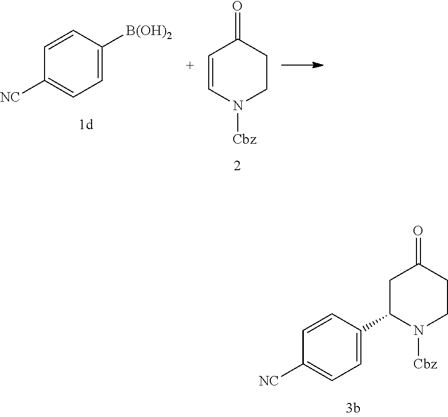 [SMILES: N#Cc1ccc(B(O)O)cc1 ; O=C1C=CN(C(=O)OCc2ccccc2)CC1 ; N#Cc1ccc([C@@H]2CC(=O)CCN2C(=O)OCc2ccccc2)cc1]

Compound 2 (23.12 g, 100 mmol) is added to the reaction flask, and tert-pentanol (115 mL) is added. The vacuum nitrogen is switched three times and then cooled to (−15° C.)-(−5° C.) under nitrogen protection. Add the catalyst (S,S)-(+)-1,2-bis[(2-methoxyphenyl)(phenyl)phosphine]ethane (1,5-cyclooctadiene) tetrafluoroborate rhodium(I) (0.2 mmol, 151 mg). After stirring evenly, slowly add the 4-cyano-phenylboronic acid 1d (17.63 g, 120 mmol dissolved in 115 mL tert-pentanol) solution using a peristaltic pump to the reaction flask. After dripping is complete, keep at (−15° C.)-(−5° C.) and stir for 3 to 4 hours. After the reaction is complete, add 5% dilute hydrochloric acid solution (230 mL) to quench the reaction, separate the phases, and extract the aqueous phase twice with ethyl acetate (115 mL). Combine the organic phases and wash once with water (115 mL), concentrate to a small volume, slowly add n-heptane (230 mL), and cool slowly to 0-5° C. After crystallization, filter the mixture to obtain the solid intermediate compound formula 3b (31.05 g, a yield of 92.4%, a purity of 99.5%, ee=99.8%).

In Example 5, the catalyst (S,S)-(+)-1,2-bis[(2-methoxyphenyl)(phenyl)phosphine]ethane (1,5-cyclooctadiene) tetrafluoroborate rhodium(I) may be replaced by (1,2-bis((2S,5S)-2,5-diethylphosphorazepane-1-yl)benzene) (1,5-cyclooctadiene) trifluorosulfonate rhodium(I), S-(+)-2,2-bis(phenylphosphine)-1,1-biphenyl (1,5-cyclooctadiene) tetrafluoroborate rhodium, 1,2-bis[(2S,5S)-2,5-diethylphosphonyl]benzene (1,5-cyclooctadiene) tetrafluoroborate rhodium(I), or 1,2-Bis [(2S,5S)-2,5-dimethylphospholanyl] benzene (cyclooctadiene) rhodium (I) tetrafluoroborate; the reaction solvent tert-pentanol may be methanol, ethanol, isopropanol, n-butanol, tert-butanol, n-pentanol, acetonitrile, tetrahydrofuran, 2-methyltetrahydrofuran, ethyl acetate, or isopropyl acetate.

### Example 6

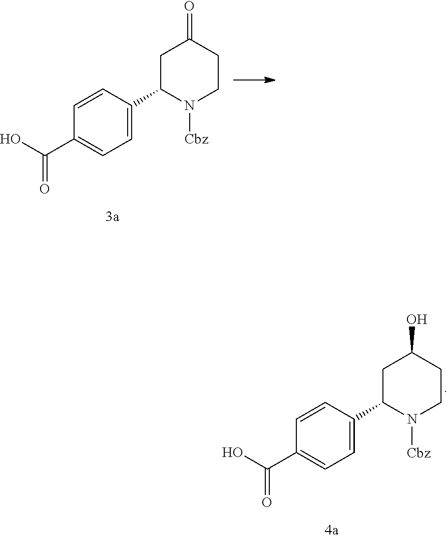 [SMILES: O=C1CCN(C(=O)OCc2ccccc2)[C@H](c2ccc(C(=O)O)cc2)C1 ; O=C(O)c1ccc([C@@H]2C[C@@H](O)CCN2C(=O)OCc2ccccc2)cc1]

Add compound 3a (3.534 g, 100 mmol) to a three-necked flask, then add 353 mL of water and 30% sodium hydroxide solution (16 mL, 120 mmol), adjust pH to 7.0-7.5 with a solution of 0.1M potassium dihydrogen phosphate and dipotassium hydrogen phosphate, add NADP (50 mg), isopropanol 34 mL, KRED-MY236 enzyme 0.3 g, and keep at 45° C.±2° C. for 28-32 hours. After the reaction is complete, add 5% sodium hydroxide solution to adjust pH to 10-12, filter out the enzyme residue, wash with a small amount of water, collect the filtrate, slowly add 0.5 M dilute hydrochloric acid to adjust pH to 3-4, and then slowly cool the temperature to 0-5° C. to precipitate white solid, filter and collect the dried product 4a (33.05 g, 91.7%, a purity of 98.6%, de.≥99.6%).

In Example 6, ketone reductase KRED-MY236 may be replaced by KRED-EW124, KRED-101, or KRED-MY352; the coenzyme nicotinamide adenine dinucleotide phosphate (NADP) may be replaced by nicotinamide adenine dinucleotide (NAD), flavin adenine dinucleotide (FAD), pyridoxal monophosphate (PLP), or mixtures thereof; isopropanol may be replaced by ethanol or glucose; water may be replaced by dimethyl sulfoxide (DMSO), N-methylpyrrolidone, acetonitrile, ethyl acetate, or isopropyl acetate, and any two of these formed mixed solution; potassium dihydrogen phosphate and dipotassium hydrogen phosphate may be replaced by phosphoric acid, disodium hydrogen phosphate, monosodium dihydrogen phosphate, or tris (hydroxymethyl) aminomethane hydrochloride, and any two of these may form a mixed buffer system.

### Example 7

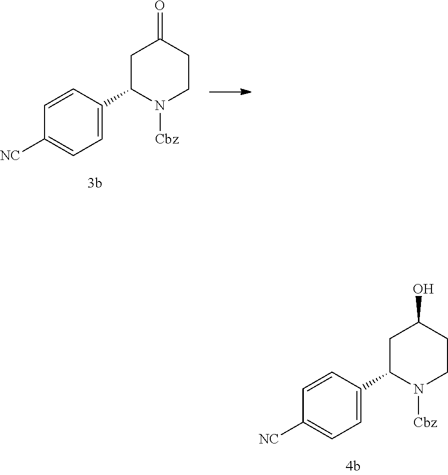 [SMILES: N#Cc1ccc([C@@H]2CC(=O)CCN2C(=O)OCc2ccccc2)cc1 ; N#Cc1ccc([C@@H]2C[C@@H](O)CCN2C(=O)OCc2ccccc2)cc1]

Add compound 3b (3.44 g, 100 mmol) to a three-necked flask, then add 334 mL of water and 100 mL of DMSO, adjust pH to 6.5-7.0 with a solution of 0.1M potassium dihydrogen phosphate and dipotassium hydrogen phosphate, add NADP (50 mg), add 34 mL of isopropanol, add 0.35 g of KRED-MY352 enzyme, and keep at 45° C.±2° C. for 28-32 hours. After the reaction is complete, add isopropyl acetate, and filter out the enzyme residue and separate the phases. The organic phase is washed with 132 ml of water, concentrated to a small volume, and n-heptane (230 ml) is slowly added and cooled to 0-5° C. After crystallization, filter the mixture to obtain the solid intermediate compound formula 4b (29.43 g, 86.7%, a purity of 99.1%, de≥99.4%).

In Example 7, dimethyl sulfoxide (DMSO) may be replaced by N-methylpyrrolidone, acetonitrile, ethyl acetate, isopropyl acetate, water or any mixture of two of these; potassium dihydrogen phosphate and dipotassium hydrogen phosphate may be replaced by phosphoric acid, disodium hydrogen phosphate, monosodium dihydrogen phosphate, or tris (hydroxymethyl) aminomethane hydrochloride, and any two of these may form a mixed buffer system; isopropanol may be replaced by ethanol or glucose; ketoreductase KRED-MY352 may be replaced by KRED-MY236, KRED-EW124, or KRED-101; the coenzyme nicotinamide adenine dinucleotide phosphate (NADP) may be replaced by nicotinamide adenine dinucleotide (NAD), flavin adenine dinucleotide (FAD), pyridoxal monophosphate (PLP), or any mixture thereof.

### Example 8

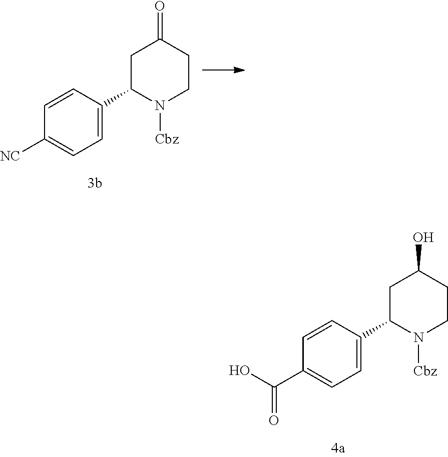 [SMILES: N#Cc1ccc([C@@H]2CC(=O)CCN2C(=O)OCc2ccccc2)cc1 ; O=C(O)c1ccc([C@@H]2C[C@@H](O)CCN2C(=O)OCc2ccccc2)cc1]

Add compound 3b (3.44 g, 100 mmol) to a three-necked flask, then add 334 mL of water and 100 mL DMSO, adjust the pH to 6.5-7.0 with a solution of 0.1M potassium dihydrogen phosphate and dipotassium hydrogen phosphate, add NADP (50 mg), add 34 mL of isopropanol, add 0.35 g of KRED-MY352 enzyme, and keep at 45° C.±2° C. for 28-32 hours. After the reaction is complete, add potassium dihydrogen phosphate solution to adjust pH to 7.2-7.5, then add NIT-MY20 cyano hydrolysis enzyme, and raise the temperature to 48-52° C. for 20 hours. After the reaction is complete, add 5% sodium hydroxide solution to adjust pH to 10-12, filter out the enzyme residue, wash with a small amount of water, collect the filtrate and slowly add 0.5 M dilute hydrochloric acid to adjust pH to 3-4, and slowly cool to 0-5° C. to precipitate white solid, filter and collect the dried product 4a (31.44 g, 87.5%, a purity of 98.9%, de≥99.5%).

In Example 8, dimethyl sulfoxide (DMSO) may be replaced by N-methylpyrrolidone, acetonitrile, ethyl acetate, isopropyl acetate, water or any mixture of two of these; potassium dihydrogen phosphate and dipotassium hydrogen phosphate may be replaced by phosphoric acid, disodium hydrogen phosphate, monosodium dihydrogen phosphate, or tris (hydroxymethyl) aminomethane hydrochloride, and any two of these may form a mixed buffer system; isopropanol may be replaced by ethanol or glucose; ketoreductase KRED-MY352 may be replaced by KRED-MY236, KRED-EW124, or KRED-101; the coenzyme nicotinamide adenine dinucleotide phosphate (NADP) may be replaced by nicotinamide adenine dinucleotide (NAD), flavin adenine dinucleotide (FAD), pyridoxal monophosphate (PLP), or any mixture thereof; cyano hydrolysis enzyme NIT-MY20 may be replaced by NIT83, NIT101, NIT139, SP409, or NIT-MY25.

### Example 9

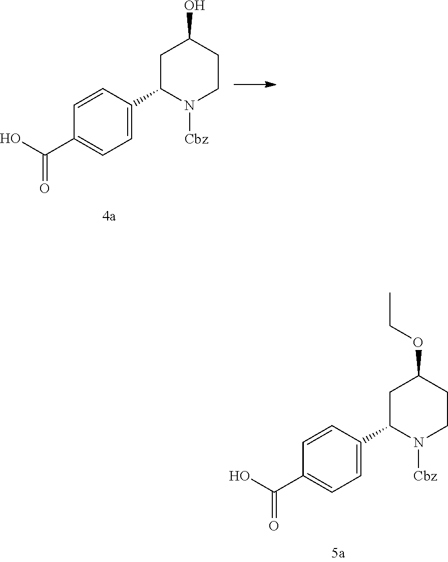 [SMILES: O=C(O)c1ccc([C@@H]2C[C@@H](O)CCN2C(=O)OCc2ccccc2)cc1 ; CCO[C@H]1CCN(C(=O)OCc2ccccc2)[C@H](c2ccc(C(=O)O)cc2)C1]

Add compound 4a (35.54 g, 100 mmol) and tetrahydrofuran (178 mL) to a three-necked flask, stir evenly, add 30% sodium hydroxide solution (66.7 g, 500 mmol), 40% tetrabutylammonium hydroxide solution (7.1 g), stir evenly, then add ethyl bromide (21.79 g, 200 mmol), heat to 40-45° C. for 6-8 hours. After the reaction is complete, cool to room temperature and add water (107 mL). Separate the solution and discard the tetrahydrofuran layer. Collect the aqueous phase and add 5% dilute hydrochloric acid to adjust pH to 3-4, and slowly cool to 0-5° C. After crystallization and filtration, wash the filter cake with a small amount of water, collecting the solid and drying to obtain compound 5a (34.36 g, a yield of 89.6%).

In Example 9, the ethylation reagent bromoethane may be replaced by diethyl sulfate, diethyl carbonate, ethyl iodide, ethoxy methanesulfonate, or ethyl p-toluenesulfonate; sodium hydroxide may be replaced by sodium methoxide, sodium ethoxide, potassium tert-butoxide, sodium tert-butoxide, LiHMDS, KHMDS, lithium hydroxide, or potassium hydroxide; tetrabutylammonium hydroxide may be omitted, or it may be replaced by potassium iodide, sodium iodide, tetrabutylammonium bromide, trimethylbenzyl ammonium chloride, or triethylbenzyl ammonium chloride; tetrahydrofuran may be replaced by DMSO, toluene, NMP, dichloromethane, 2-methyltetrahydrofuran, or a mixture of these solvents with water.

### Example 10

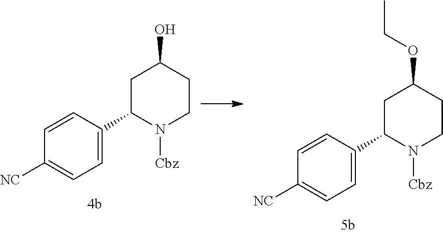 [SMILES: N#Cc1ccc([C@@H]2C[C@@H](O)CCN2C(=O)OCc2ccccc2)cc1 ; CCO[C@H]1CCN(C(=O)OCc2ccccc2)[C@H](c2ccc(C#N)cc2)C1]

Add compound 4b (33.64 g, 100 mmol) and NMP (168 mL) to a three-necked flask and stir evenly. Add potassium tert-butoxide (22.44 g, 200 mmol), potassium iodide (3.32 g, 2 mmol), stir evenly. Add bromoethane (13.08 g, 120 mmol) and heat to 50-55° C. for 8-10 hours. After the reaction is complete, the mixture is cooled to room temperature and water (202 mL) is added. Ethyl acetate (101 mL) is added and extracted twice. The organic phase is washed once with 101 mL of water. The mixture is concentrated and separated by column chromatography to obtain the target product 5b (33.35 g, a yield of 91.5%).

In Example 10, the ethylating reagent bromoethane may be replaced by diethyl carbonate, diethyl sulfate, ethyl iodide, ethoxymethyl sulfonate, or ethoxytoluene sulfonate; potassium tert-butoxide may be replaced by sodium methoxide, sodium ethanolamine, sodium tert-butoxide, LiHMDS, KHMDS, lithium hydroxide, or sodium hydroxide; potassium iodide may not be added, or it may be replaced by sodium iodide, tetrabutylammonium bromide, tetrabutylammonium hydroxide, trimethylbenzyl ammonium chloride, or triethylbenzyl ammonium chloride; NMP may be replaced by tetrahydrofuran, DMSO, toluene, dichloromethane, or 2-methyltetrahydrofuran.

### Example 11

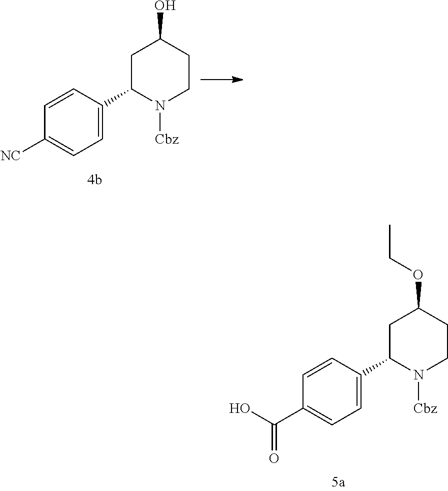 [SMILES: N#Cc1ccc([C@@H]2C[C@@H](O)CCN2C(=O)OCc2ccccc2)cc1 ; CCO[C@H]1CCN(C(=O)OCc2ccccc2)[C@H](c2ccc(C(=O)O)cc2)C1]

Add compound 4b (33.64 g, 100 mmol) and toluene (168 mL) to a three-necked flask, stir evenly, add 30% potassium hydroxide solution (91.8 g, 500 mmol), 40% tetrabutylammonium hydroxide solution (6.6 g), stir evenly and then add diethyl carbonate (59.07 g, 500 mmol), heat to 75-80° C. for 12-16 hours. After the reaction is complete, cool to room temperature and add water (101 mL). Separate the solution and discard the toluene layer. Collect the aqueous phase and add 5% dilute hydrochloric acid to adjust pH to 3-4, and slowly cool to 0-5° C. After crystallization and filtration, wash the filter cake with a small amount of water, collect the solid and dry it to obtain compound 5a (33.44 g, a yield of 87.2%).

In Example 11, the ethylation reagent diethyl carbonate may be replaced by ethyl bromide, diethyl sulfate, ethyl iodide, ethoxymethyl sulfonate, or ethoxymethyl p-toluenesulfonate; potassium hydroxide may be replaced by sodium methoxide, sodium ethoxide, potassium tert-butoxide, sodium tert-butoxide, LiHMDS, KHMDS, lithium hydroxide, or sodium hydroxide; tetrabutylammonium hydroxide may not be added, and may also be replaced by potassium iodide, sodium iodide, tetrabutylammonium bromide, trimethylbenzyl ammonium chloride, or triethylbenzyl ammonium chloride; toluene may be replaced by tetrahydrofuran, DMSO, NMP, dichloromethane, 2-methyltetrahydrofuran, or their mixed solvent systems with water.

### Example 12

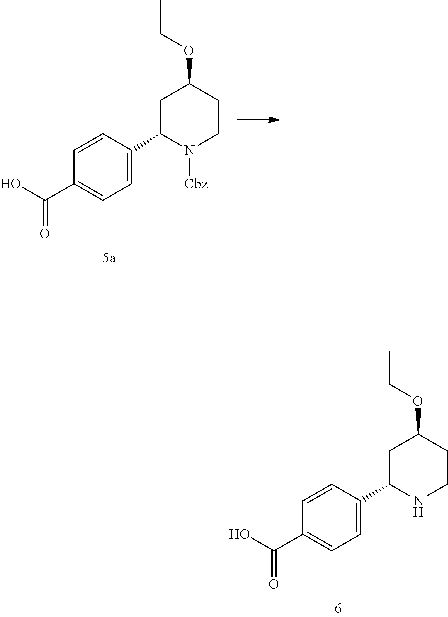 [SMILES: CCO[C@H]1CCN(C(=O)OCc2ccccc2)[C@H](c2ccc(C(=O)O)cc2)C1 ; CCO[C@H]1CCN[C@H](c2ccc(C(=O)O)cc2)C1]

Compound 5a (38.34 g, 100 mmol) is added to the hydrogenation flask and methanol (383 mL) is added to dissolve it, 3% palladium on carbon (76.7 mg) is added. Replace the air with hydrogen three times under vacuum. Then increase the pressure to 0.35-0.40 MPa and maintain the internal temperature at 35-40° C. for a hydrogenation reaction lasting 20-24 hours. After the reaction is complete, cool to room temperature, filter out palladium on carbon, collect filtrate, and concentrate under reduced pressure to distill off part of methanol. Heat to 50-55° C., add methyl tert-butyl ether (230 mL), and stir evenly at 50-55° C. Then slowly cool to 0-5° C. and filtered. Add a small amount of cold methyl tert-butyl ether to wash the filter cake. Collect the solid and dry the compound 6 (23.83 g, a yield of 95.6%).

In embodiment 12, palladium on carbon may be replaced by palladium hydroxide; the solvent methanol of the reaction may be replaced by ethanol, isopropanol, tetrahydrofuran, ethyl acetate, or isopropyl acetate.

### Example 13

### Preparation of (2S,4S)-benzyl 4-ethoxy-2-(4-(methoxycarbonyl)phenyl) piperidine-1-carboxylate

Add compound 5a (38.34 g, 100 mmol) and acetonitrile (190 mL) to a three-necked flask, stir evenly, add Potassium carbonate (20.73 g, 150 mmol), stir evenly, then add Methyl p-toluenesulfonate (24.21 g, 130 mmol), stir at RT for 5-8 hours. After the reaction is complete, add water (383 mL) and stir for 1-2 h. Filtration, wash the filter cake with a small amount of water, collecting the solid and drying to obtain compound (2S,4S)-benzyl 4-ethoxy-2-(4-(methoxycarbonyl)phenyl) piperidine-1-carboxylate (37.24 g, a yield of 93.7%).

In Example 13, the methylation reagent Methyl p-toluenesulfonate may be replaced by dimethyl sulfate, dimethyl carbonate, methyl iodide, or methoxy methanesulfonate; Potassium carbonate may be replaced by sodium methoxide, sodium ethoxide, potassium tert-butoxide, sodium tert-butoxide, LiHMDS, KHMDS, sodium carbonate, sodium hydroxide, lithium hydroxide, or potassium hydroxide; Acetonitrile may be replaced by DMF, DMAC, THE, Acetone, DMSO, toluene, NMP, dichloromethane, 2-methyltetrahydrofuran, or a mixture of these solvents with water.

### Examples 14

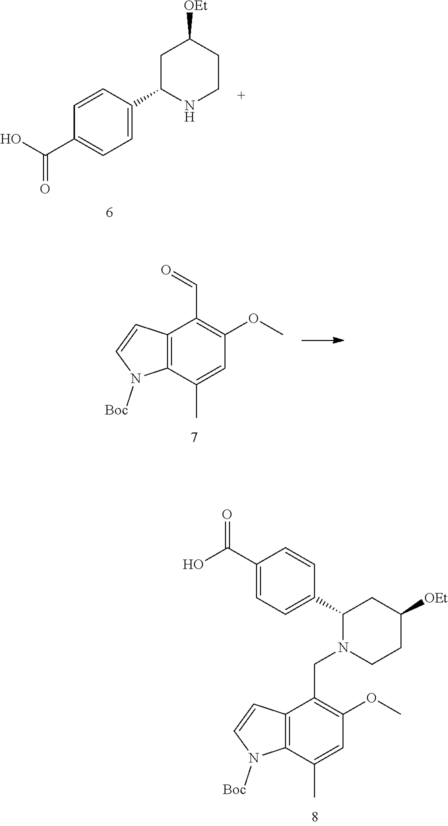 [SMILES: CCO[C@H]1CCN[C@H](c2ccc(C(=O)O)cc2)C1 ; COc1cc(C)c2c(ccn2C(=O)OC(C)(C)C)c1C=O ; CCO[C@H]1CCN(Cc2c(OC)cc(C)c3c2ccn3C(=O)OC(C)(C)C)[C@H](c2ccc(C(=O)O)cc2)C1]

Compound 6 (24.93 g, 100 mmol), compound 7 (28.93 g, 100 mmol), and dichloromethane (125 mL) are added to the reaction flask. Stir at 20-30° C. for 30 minutes and cool to 0-5° C. Add acetic acid (7.20 g, 120 mmol), sodium triacetoxyborohydride (42.39 g, 200 mmol) to the mixture in batches. Stir at 0-5° C. for 2-3 hours. Raise the temperature of the reaction mixture to room temperature and keep it at this temperature for 4 hours. After the reaction is complete, add 125 mL of 0.5% dilute hydrochloric acid to quench the reaction. Separate the solution, and extract the aqueous phase twice with dichloromethane (125 mL). Wash the organic phase once with water (62 mL), concentrate it to a small volume, add isopropanol (62 mL) and stir to dissolve it. Heat to 55-60° C., slowly add 250 mL of water and reduce to 0-5° C. After crystallization and filtration, wash the filter cake with a small amount of water, collect the solid and dry it to obtain intermediate 8 (48.07 g, a yield of 92.0%)

In Example 14, sodium triacetoxyborohydride may be replaced by sodium borohydride, lithium borohydride, potassium borohydride, sodium cyanoborohydride, triethylsilane, or 1,1,3,3-tetramethyldisiloxane; acetic acid may not be added, or may be replaced by titanium tetrachloride or tetraisopropyl titanate; dichloromethane may be replaced by 1,2-dichloroethane, acetonitrile, tetrahydrofuran, 1,4-dioxane, or toluene.

### Example 15

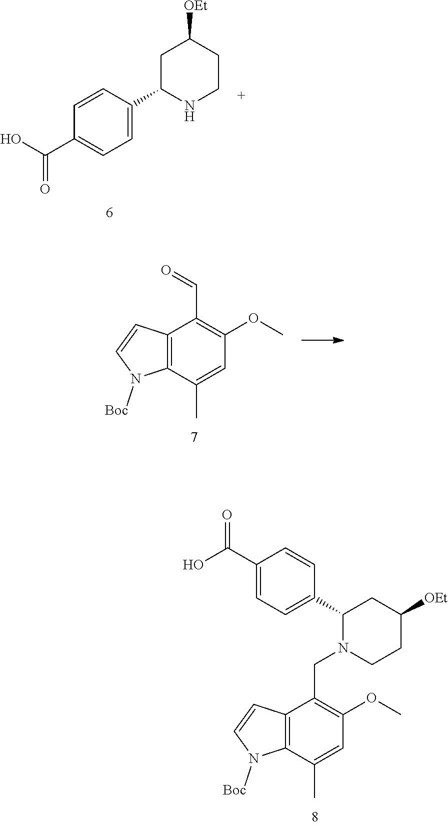 [SMILES: CCO[C@H]1CCN[C@H](c2ccc(C(=O)O)cc2)C1 ; COc1cc(C)c2c(ccn2C(=O)OC(C)(C)C)c1C=O ; CCO[C@H]1CCN(Cc2c(OC)cc(C)c3c2ccn3C(=O)OC(C)(C)C)[C@H](c2ccc(C(=O)O)cc2)C1]

Compound 6 (24.93 g, 100 mmol), compound 7 (28.93 g, 100 mmol), and acetonitrile (125 mL) are added to the reaction flask. Stir at 20-30° C. for 30 minutes and cool to 0-5° C. Add tetraisopropyl titanate (34.11 g, 120 mmol), and sodium triacetoxyborohydride (42.39 g, 200 mmol) in batches at 0-5° C. Stir at 0-5° C. for 2-3 hours, raise to room temperature and stir at this temperature for 4 hours. After the reaction is complete, add 125 mL of 0.5% dilute hydrochloric acid to quench the reaction, separate the phases, and extract the aqueous phase twice with dichloromethane (125 mL). Wash the organic phase once with water (62 mL), concentrate to a small volume, add isopropanol (62 mL) and stir to dissolve, and heat to 55-60° C., slowly add 250 mL of water and reduce to 0-5° C. After crystallization and filtration, wash the filter cake with a small amount of water, collect the solid and dry to obtain intermediate 8 (46.62 g, a yield of 89.2%)

In Example 15, sodium triacetoxyborohydride may be replaced by sodium borohydride, lithium borohydride, potassium borohydride, sodium cyanoborohydride, triethylsilane, or 1,1,3,3-tetramethyldisiloxane; tetraisopropyl titanate may be omitted, or titanium tetrachloride or acetic acid may be used instead; acetonitrile may be replaced by dichloromethane, 1,2-dichloroethane, tetrahydrofuran, 1,4-dioxane, or toluene.

### Example 16

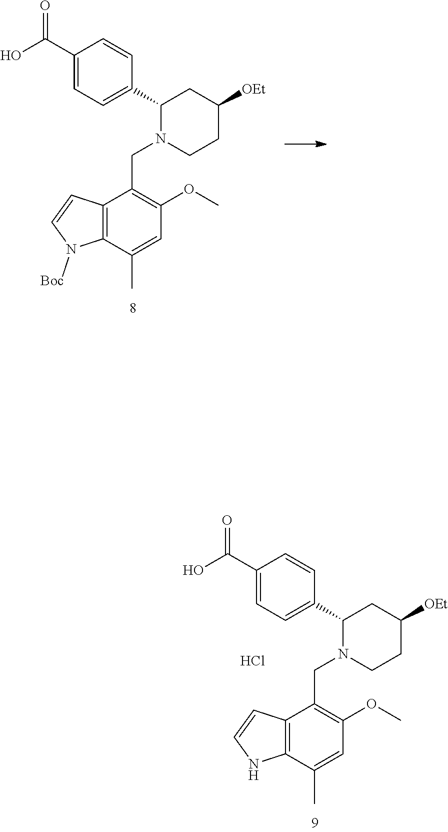 [SMILES: CCO[C@H]1CCN(Cc2c(OC)cc(C)c3c2ccn3C(=O)OC(C)(C)C)[C@H](c2ccc(C(=O)O)cc2)C1 ; CCO[C@H]1CCN(Cc2c(OC)cc(C)c3[nH]ccc23)[C@H](c2ccc(C(=O)O)cc2)C1 ; Cl]

Add intermediate 8 (52.26 g, 100 mmol) and isopropanol (161 mL) to the reaction flask, stir evenly, and heat to 55-60° C., then add 36% concentrated hydrochloric acid (15.2 g, 150 mmol). Stir at 55-60° C. for 2-3 hours. Slowly cool to room temperature, pulp, filter, and wash with a small amount of isopropanol, and dry to obtain the product 9 iptacopan hydrochloride (41.10 g, a purity of 99.8%, a yield of 89.4%).

In embodiment 16, isopropanol may be replaced by methanol, ethanol, n-butanol, tert-butanol, tert-pentanol, ethyl acetate, isopropyl acetate, tetrahydrofuran, 2-methyltetrahydrofuran, 1,4-dioxane, methyl tert-butyl ether, isopropyl ether, acetone, acetonitrile, toluene or water, or any mixture of two of these.

### Example 17

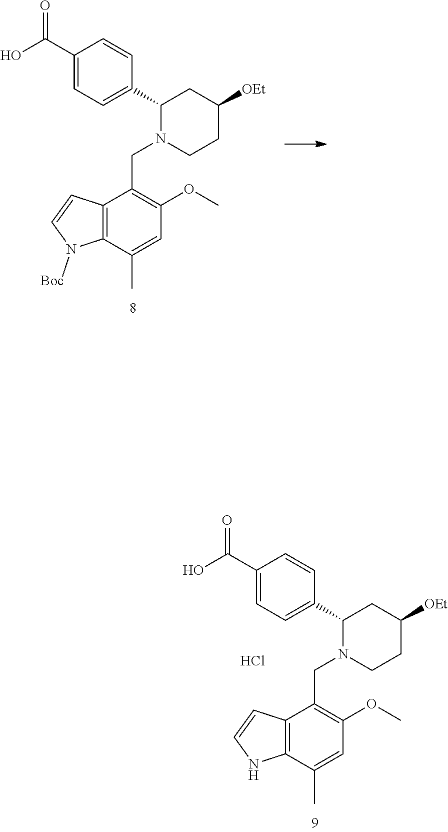 [SMILES: CCO[C@H]1CCN(Cc2c(OC)cc(C)c3c2ccn3C(=O)OC(C)(C)C)[C@H](c2ccc(C(=O)O)cc2)C1 ; CCO[C@H]1CCN(Cc2c(OC)cc(C)c3[nH]ccc23)[C@H](c2ccc(C(=O)O)cc2)C1 ; Cl]

Add intermediate 8 (52.26 g, 100 mmol) and n-butanol (161 mL) to the reaction flask, stir evenly, and heat to 55-60° C. Stir for 6-8 hours until the raw material completely disappears. Slowly cool to room temperature, and add 30% methanolic hydrochloric acid solution (14.58 g, 120 mmol), then slowly cool to 0-5° C. for slurrying. Filter and wash with a small amount of n-butanol and dry to obtain product 9 iptacopan hydrochloride (43.42 g, a purity of 99.9%, a yield of 94.5%).

In embodiment 17, n-butanol may be replaced by a mixture of isopropanol, methanol, ethanol, tert-butanol, tert-pentanol or water and any two of them.

### Example 18

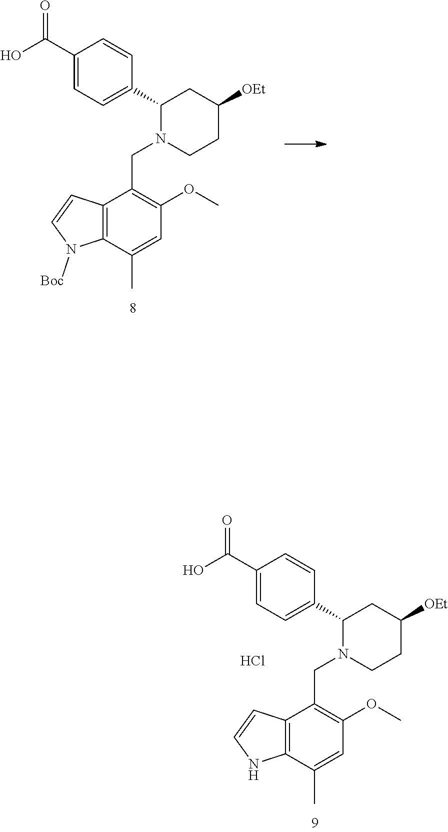 [SMILES: CCO[C@H]1CCN(Cc2c(OC)cc(C)c3c2ccn3C(=O)OC(C)(C)C)[C@H](c2ccc(C(=O)O)cc2)C1 ; CCO[C@H]1CCN(Cc2c(OC)cc(C)c3[nH]ccc23)[C@H](c2ccc(C(=O)O)cc2)C1 ; Cl]

Add intermediate 8 (52.26 g, 100 mmol) and ethanol (161 mL) to the reaction flask, stir evenly, then add p-toluenesulfonic acid (20.66 g, 120 mmol), heat to 55-60° C. Stir for 2-3 hours until the raw material completely disappears, add 30% ethanolic hydrochloric acid solution (14.58 g, 120 mmol), and slowly cool to 0-5° C. for slurrying. Filter and wash with a small amount of ethanol and dry to obtain product 9 iptacopan hydrochloride (42.45 g, a purity of 99.8%, a yield of 92.3%).

In Example 18, p-toluenesulfonic acid may be replaced by acetic acid, citric acid, tartaric acid, methanesulfonic acid, or trifluoromethanesulfonic acid; n-butanol may be replaced by isopropanol, methanol, ethanol, tert-butanol, tert-pentanol, ethyl acetate, isopropyl acetate, tetrahydrofuran, 2-methyltetrahydrofuran, 1,4-dioxane, methyl tert-butyl ether, isopropyl ether, acetone, acetonitrile, toluene, or water, or any mixture of two of these.

What is claimed is:

1. A process for preparation of Iptacopan, comprising the steps of:
(a) a compound 6 and a compound 7 undergoing a reductive amination reaction to get an intermediate compound 8; 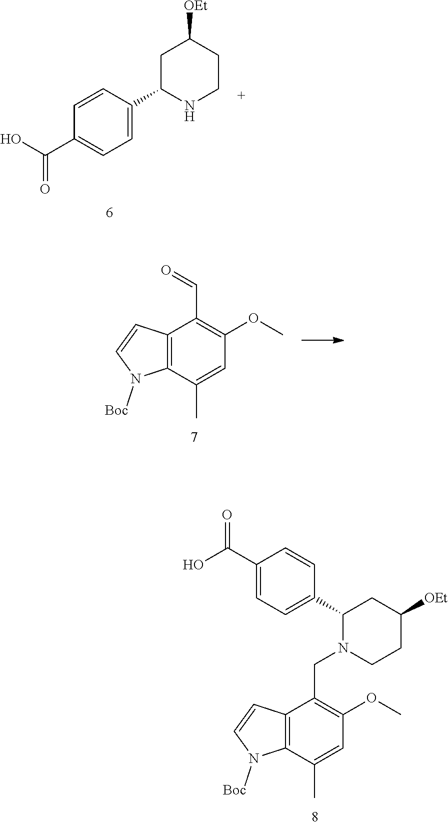 [SMILES: CCO[C@H]1CCN[C@H](c2ccc(C(=O)O)cc2)C1 ; COc1cc(C)c2c(ccn2C(=O)OC(C)(C)C)c1C=O ; CCO[C@H]1CCN(Cc2c(OC)cc(C)c3c2ccn3C(=O)OC(C)(C)C)[C@H](c2ccc(C(=O)O)cc2)C1]
(b) the compound 8 undergoing a de-protection reaction to remove a Boc protection group and a salification reaction with hydrochloric acid to obtain a compound 9 Iptacopan hydrochloride;

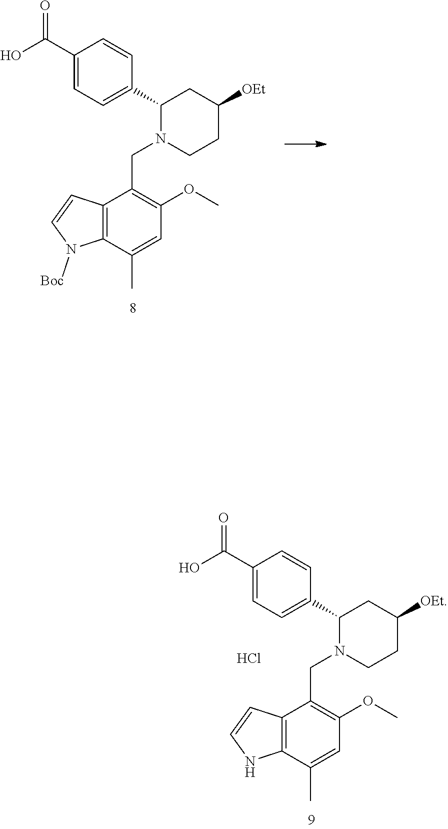 [SMILES: CCO[C@H]1CCN(Cc2c(OC)cc(C)c3c2ccn3C(=O)OC(C)(C)C)[C@H](c2ccc(C(=O)O)cc2)C1 ; CCO[C@H]1CCN(Cc2c(OC)cc(C)c3[nH]ccc23)[C@H](c2ccc(C(=O)O)cc2)C1 ; Cl]

2. The process for the preparation of Iptacopan according to claim 1, wherein
in step (a), a reducing reagent used in the reductive amination reaction is selected from sodium borohydride, lithium borohydride, potassium borohydride, sodium triacetoxyborohydride, sodium cyanoborohydride, triethylsilane, or 1,1,3,3-tetramethyldisiloxane; without adding Lewis acid or with the addition of Lewis acid, wherein the Lewis acid is selected from acetic acid, titanium tetrachloride, or tetraisopropyl titanate; and a reaction solvent is selected from dichloromethane, 1,2-dichloroethane, acetonitrile, tetrahydrofuran, 1,4-dioxane, or toluene; and
in the step (b), the compound 9 is obtained in three ways including adding hydrochloric acid to perform the de-protection reaction by removing Boc protection group and the salification reaction in one-pot, or by directly removing Boc protection without the acid and then performing the salification reaction with hydrochloric acid, or by removing Boc protection with an organic acid and then performing salification reaction with hydrochloric acid; the organic acid is selected from acetic acid, trifluoroacetic acid, citric acid, tartaric acid, methanesulfonic acid, p-toluenesulfonic acid, or trifluoromethanesulfonic acid; and a reaction solvent is selected from methanol, ethanol, isopropanol, n-butanol, tert-butanol, isopentanol, tert-pentanol, ethyl acetate, isopropyl acetate, tetrahydrofuran, 2-methyltetrahydrofuran, 1,4-dioxane, methyl tert-butyl ether, isopropyl ether, acetone, acetonitrile, toluene, or water, or any mixture of two of these solvents.

3. A process for preparation of an intermediate compound 6 of iptacopan, comprising the following steps:
(a) a compound 4 undergoing an alkylation reaction with an ethylating reagent in the presence of base to obtain a compound 5; 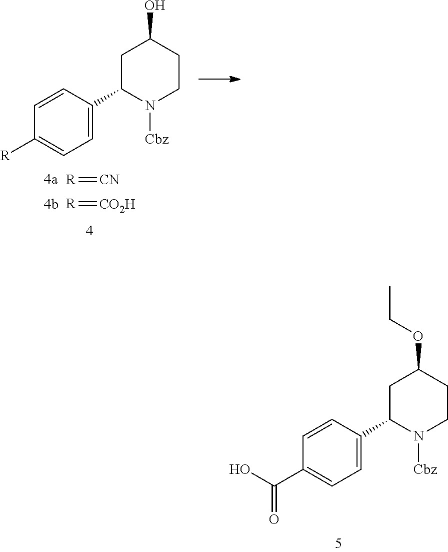
(b) the compound 5 undergoing a hydrogenation reaction under palladium catalyst to remove Cbz protection group to obtain the intermediate compound 6;

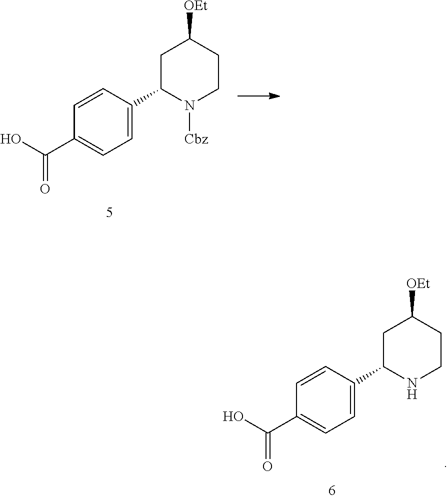 [SMILES: CCO[C@H]1CCN(C(=O)OCc2ccccc2)[C@H](c2ccc(C(=O)O)cc2)C1 ; CCO[C@H]1CCN[C@H](c2ccc(C(=O)O)cc2)C1]

4. The process for the preparation of the intermediate compound 6 of iptacopan according to claim 3, wherein in the alkylation reaction of step (a), the ethylating reagent is selected from diethyl sulfate, diethyl carbonate, ethyl iodide, ethyl bromide, ethyl methanesulfonate, or ethyl p-toluenesulfonate; the base is selected from sodium methoxide, sodium ethoxide, potassium tert-butoxide, sodium tert-butoxide, LiHMDS, KHMDS, sodium hydroxide, lithium hydroxide, or potassium hydroxide; without adding a catalyst or with the addition of a catalyst, wherein the catalyst is selected from potassium iodide, sodium iodide, tetrabutylammonium bromide, tetrabutylammonium hydroxide, trimethylbenzyl ammonium chloride, or triethylbenzyl ammonium chloride; and a reaction solvent is selected from DMSO, toluene, NMP, dichloromethane, tetrahydrofuran, 2-methyltetrahydrofuran, or any mixture of two of these solvents or water; and
in step (b), the palladium catalyst is selected from palladium on carbon or palladium hydroxide; and a reaction solvent is selected from methanol, ethanol, isopropanol, tetrahydrofuran, ethyl acetate, or isopropyl acetate.

5. The process for the preparation of the intermediate compound 6 of iptacopan according to claim 4, wherein in the compound 4, R is CN, a cyanide hydrolysis reaction is directly performed by adding water and heating or heating in an original alkaline aqueous mixture, and in the compound 5, R is COOH.

6. A process for preparation of an intermediate compound 4 of iptacopan, comprising the following steps:
(a) a compound 1 and a compound 2 undergoing an asymmetric addition reaction to obtain an intermediate compound 3 under catalysis of a chiral phosphine ligand metal complex catalyst; 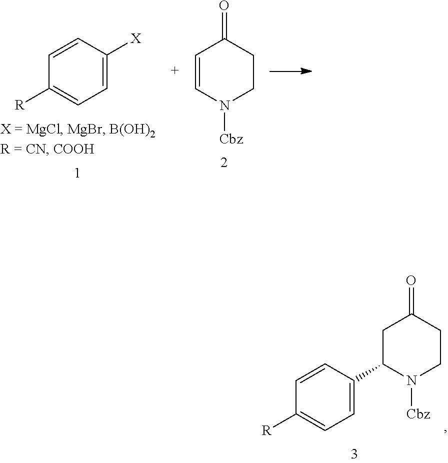
(b) the compound 3 undergoing a carbonyl reduction reaction to obtain the intermediate compound 4 under the catalysis of a reductase;

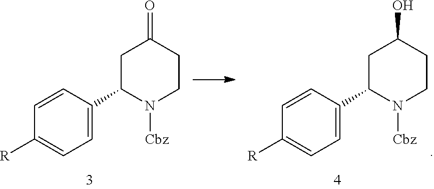

7. The process for the preparation of the intermediate Compound 4 of iptacopan according to claim 6, wherein in the asymmetric addition reaction of step (a), the chiral phosphine ligand metal complex catalyst is a complex formed by a metal catalyst and a chiral phosphine ligand, which is used directly or generated in situ to participation in the asymmetric addition reaction, the metal catalyst is selected from cuprous bromide, cuprous iodide, cuprous chloride, palladium acetate, palladium trifluoroacetate, palladium dichloride, sodium tetrachloropalladate, chlorobis (ethylene) rhodium (I) dimer, or bis(1,5-cyclooctadiene) rhodium(I) tetrafluoroborate;
when X is MgCl or MgBr, the compound 1 is obtained by an exchange of 4-bromobenzoic acid, 4-iodobenzoic acid, 4-bromobenzonitrile, or 4-iodobenzonitrile with isopropyl magnesium chloride, isopropylmagnesium chloride-lithium chloride complex, or isopropyl magnesium bromide, or any combination of these with n-butyl lithium; the catalyst is a complex formed in situ from copper metal catalyst and chiral phosphine ligand; the copper metal catalyst is selected from cuprous bromide, cuprous iodide, or cuprous chloride, and the preferred chiral phosphine ligand is selected from 1,2-bis((2S,5S)-2,5-diethylphospholane-1-yl)benzene, S-(−)-2,2′-bis(diphenylphosphino)-1,1′-binaphthalene, 1,2-bis((2S,5S)-2,5-dimethylphospholane)benzene, 1,2-bis[(2S,5S)-2,5-diethylphospholane]benzene, 1,2-bis[(2S,5S)-2,5-dimethylphospholane]ethane, 1,2-bis((2S,5S)-2,5-diphenylphospholane)ethane or (S,S)-bis[(2-methoxyphenyl)phenylphosphino]ethane; a reaction solvent is selected from dichloromethane, 1,2-dichloroethane, toluene, tetrahydrofuran, or 2-methyltetrahydrofuran, or any mixture of two of these solvents;
when X is B(OH)₂, a preferred catalyst is selected from (1,2-bis((2S,5S)-2,5-diethylphosphorazepane-1-yl)benzene) (1,5-cyclooctadiene) trifluoromethanesulfonate rhodium(I), S-(+)-2,2-bis(diphenylphosphine)-1,1-bis-naphthalene (1,5-cyclooctadiene) tetrafluoroborate rhodium, 1,2-bis[(2S,5S)-2,5-diethylphosphonyl]benzene (1,5-cyclooctadiene) tetrafluoroborate rhodium(I), 1,2-bis[(2S,5S)-2,5-dimethylphosphorazepane-1,3-phenylene]benzene (cyclooctadiene) tetrafluoroborate rhodium(I), or (S,S)-(+)-1,2-bis[(2-methoxyphenyl)(phenyl)phosphine]ethane (1,5-cyclooctadiene) tetrafluoroborate rhodium(I); the reaction solvent is selected from methanol, ethanol, isopropanol, n-butanol, tert-butanol, n-pentanol, tert-pentanol, acetonitrile, tetrahydrofuran, 2-methyltetrahydrofuran, ethyl acetate or isopropyl acetate.

8. The process for the preparation of the compound 4 of iptacopan according to claim 6, wherein in the carbonyl reduction reaction of step (b), the reductase is selected from ketoreductase KRED, alcohol dehydrogenase, isopropanol dehydrogenase, or glucose dehydrogenase (GDH), or any combination of two of these; the ketoreductase is selected from KRED-EW124, KRED-101, KRED-MY236, or KRED-MY352; the coenzyme used is selected from nicotinamide adenine dinucleotide (NAD), nicotinamide adenine dinucleotide phosphate (NADP), flavin adenine dinucleotide (FAD), pyridoxal monophosphate (PLP), or mixtures of them; the reducing reagent is selected from isopropanol, ethanol, or glucose; a reaction solvent is selected from dimethyl sulfoxide (DMSO), N-methylpyrrolidone, acetonitrile, ethyl acetate, isopropyl acetate, water or any mixture of two of these solvents; the buffer reagent is selected from phosphoric acid, potassium dihydrogen phosphate, dipotassium hydrogen phosphate, disodium hydrogen phosphate, monosodium dihydrogen phosphate, tris (hydroxymethyl) aminomethane hydrochloride, or any mixture buffer solution of two of these reagents.

9. The process for the preparation of the intermediate compound 4 of iptacopan according to claim 8, wherein in step (b), when R is CN, the carbonyl group is first reduced under the catalysis of a reductase, followed by the one-pot addition of a cyano hydrolysis enzyme to hydrolyze the cyano group into a carboxylic acid: 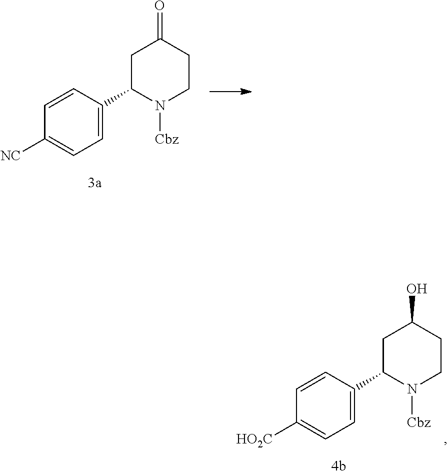 [SMILES: N#Cc1ccc([C@@H]2CC(=O)CCN2C(=O)OCc2ccccc2)cc1 ; O=C(O)c1ccc([C@@H]2C[C@@H](O)CCN2C(=O)OCc2ccccc2)cc1]
the one-pot cyano hydrolysis reaction involves adjusting a reaction mixture to pH of 5-9 using PBS buffer solution after the carbonyl reduction reaction is complete, cyano hydrolysis enzyme is then added to convert the cyano group into a carboxylic acid group in one pot, and the cyano hydrolysis enzyme is selected from NIT83, NIT101, NIT139, SP409, NIT-MY20, or NIT-MY25.
